(19) United States
(12) Patent Application Publication
(10) Pub. No.: US 2008/0293087 A1
(43) Pub. Date: Nov. 27, 2008

(54) SULFONYLUREA RECEPTOR SHORT FORMS FROM MITOCHONDRIA AND USES THEREOF

(76) Inventors: Jonathan C. Makielski, Fitchburg, WI (US); Nian-Qing Shi, Middleton, WI (US); Bin Ye, Middleton, WI (US)

Correspondence Address: QUARLES & BRADY LLP, 33 E. MAIN ST, SUITE 900, P.O. BOX 2113, MADISON, WI, 53701-2113, US

(21) Appl. No.: 11/931,916

(22) Filed: Oct. 31, 2007

Related U.S. Application Data
(60) Provisional application No. 60/855,527, filed on Oct. 31, 2006.

(51) Int. Cl. C12Q 1/02; C07H 21/00; C07K 14/00; C12N 15/63; C12N 5/00

(52) U.S. Cl. 435/29; 536/23.1; 530/350; 435/320.1; 435/325

(57) ABSTRACT

The present invention relates to isolated sulfonylurea receptor polynucleotides and polypeptides, as well as vectors and cells lines containing the polynucleotides and polypeptides. The present invention also relates to methods of using cell lines containing the polynucleotides and polypeptides to identify agents that are useful in ischemic preconditioning.

### CROSS-REFERENCE TO RELATED APPLICATIONS

This application claims the benefit of U.S. Provisional Patent Application No. 60/855,527, filed Oct. 31, 2006, incorporated herein by reference as if set forth in its entirety.

### STATEMENT REGARDING FEDERALLY SPONSORED RESEARCH OR DEVELOPMENT

This invention was made with United States government support awarded by the following agency: N1H HL-57414. The United States has certain rights in this invention.

### BACKGROUND

The invention relates generally to isolated polynucleotides and polypeptides, and more particularly to isolated polynucleotides and polypeptides useful in connection with ischemic preconditioning (IPC) and protection from reperfusion injury.

Ischemia is a condition in which a tissue experiences an absolute or a relative deoxygenation when oxygen demand exceeds oxygen delivery. Tissues sensitive to ischemia include, but are not limited to, heart and brain. Reperfusion injury can occur when blood flow is restored to a tissue after an ischemic episode, and is characterized by inflammation and oxidative damage, rather than a return of normal cellular processes. Reperfusion injury therefore can permanently damage the myocardium, which leads to cardiac dysfunction and even repeated myocardial infarction.

IPC is a phenomenon whereby single or multiple brief periods of ischemia protect a tissue from subsequent, prolonged ischemia. IPC was first described by Murry el al., who demonstrated that repeated and short cycles of ischemia (e.g., circumflex artery occlusion and reperfusion) reduced infarct size resulting from prolonged ischemia. Murry C, et al., “Preconditioning with ischemia: a delay of lethal cell injury in ischemic myocardium,” Circulation 74:1124-1136 (1986). IPC protects not only the heart, but also the brain. Stone T, “Pre-conditioninig protection in the brain,” Br. J. Pharmacol. 140:229-230 (2003); and Kitagawa K, et al., “Ischemic tolerance phenomenon found in the brain,” Brain Res. 528:21-24 (1990). IPC is hypothesized to preserve cellular energy stores and to suppress deleterious downstream events, such as cellular calcium overload. IPC has two beneficial phases. The first phase, called acute preconditioning, occurs early and lasts approximately two to three hours after an ischemic episode. The second phase, called delayed preconditioning, occurs about one day later and lasts approximately three days.

ATP-sensitive potassium channels (K_{ATP}) may be trigger, mediators and end effectors of IPC and may decrease cytosolic and mitochondrial calcium overload. Yellon D & Downey J, “Preconditioning the myocardium: from cellular physiology to clinical cardiology,” Physiol. Rev. 83: 1113-1151 (2003). Under physiological conditions, K_{ATP} are inhibited by intracellular ATP, but open in response to various intracellular signals. Gross G & Auchampach J, “Blockade of ATP-sensitive potassium channels prevents myocardial preconditioning in dogs,” Circ. Res. 70:223-233 (1992); and Ashcroft S & Ashcroft F, “Properties and functions of ATP-sensitive K-channels,” Cell. Signal. 2:197-214 (1990).

Two types of K_{ATP} are thought to be involved in IPC. The first K_{ATP} is CellK_{ATP}, which is associated with the plasma membrane. cellK_{ATP} is a hetero-octamer that contains, in a 4:4 ratio, (1) a pore-forming inwardly rectifying potassium channel (K_{IR}6.x) subunit, and (2) a regulatory sulfonylurea receptor (SUR) subunit. The second K_{ATP} is mitoK_{ATP}, which is associated with the inner mitochondrial membrane. Although the stricture of cellK_{ATP} is known, the structure of mitoK_{ATP} is less defined. mitoK_{ATP} is thought to contain components similar to those of cellK_{ATP}, particularly SUR2.

SUR, an ATP-binding cassette (ABC) transporter, exists in at least two isoforms, a high-affinity receptor, SUR1 (˜177 kDa), and a low-affinity receptor, SUR2 (˜174 kDa). Each SUR isoform exists as multiple alternative splice variants present at various levels in a variety of cell types. SURs confer upon K_{ATP} a sensitivity to sulfonylureas (i.e., channel openers) and other activating nucleotides. They also account for major pharmacological differences between K_{ATP} in various tissues. Babenko A, el al., “A view of SUR/K_{IR}6.X, KATP channel,” Ann. Rev. Physiol. 60:667-687 (1998). SUR sequences are available for human, rat and mouse, and show about 90% identity at an amino acid level. Aguilar-Bryan L, el al., “Cloning of the β cell high-affinity sulfonylurea receptor: a regulator of insulin secretion,” Science 268:423-426 (1995); and Isomoto S, el al., “A novel sulfonylurea receptor forms with BIR (K_{IR}6.2) a smooth muscle type ATP-sensitive K+ channel,” J. Biol. Chem. 271:24321-24324 (1996), each of which is incorporated herein by reference as if set forth in its entirety.

Of particular interest herein is the low-affinity receptor, SUR2. SUR2 includes two nucleotide-binding domains and seventeen transmembrane helices that form three transmembrane domains. In cardiac cells and vascular smooth muscle cells, SUR2 is encoded by two splice variants (SUR2A and SUR2B), which differ by an alternative use of the last exon (exon 38) in the carboxy-terminus. Shi N, et al., “Function and distribution of the SUR isoforms and splice variants,” J. Mol. Cell. Cardiol. 39:51-60 (2005), incorporated herein by reference as if set forth in its entirety.

With respect to mitoK_{ATP}, Singh et al. showed that cardiac mitochondria contain a long form of SUR2 (˜140 kDa), and Szewczyk et al. showed that cardiac mitochondria contain a short form of SUR2 (˜28 kDa). Singh H, et al., “Distribution of Kir6.0 and SUR2 ATP-sensitive potassium channel subunits in isolated ventricular myocytes,” J. Mol. Cell. Cardiol. 35:445-459 (2003); and Szewczyk A, et al., “The mitochondrial sulfonylurea receptor: identification and characterization,” Biochem. Biophys. Res. Commun. 230:611-615 (1997), each of which is incorporated herein by reference as if set forth in its entirety. Putative mitoK_{ATP} structural proteins ranging in size from 54 kDa to 63 kDa have been purified from mitochondria using ATP-affinity chromatography, followed by K_{ATP} activity reconstitution assays in proteoliposomes. Mironova G, et al., “Protein from beef heart mitochondria inducing the potassium channel conductivity of bilayer lipid membrane,” Biofizika 26:451-457 (1981); Paucek P, et al., “Reconstitution and partial purification of the glibenclamide-sensitive, ATP-dependent K+ channel from rat liver and beef heart mitochondria,” J. Bio. Chem. 267:26062-26069 (1992); Bajgar R, et al., “Identification and properties of a novel intracellular (mitochondrial) ATP-sensitive potassium channel in brain,” J. Biol. Chem. 276:33369-33374 (2001); and Mironova G, et al. “Functional distinctions between the mitochondrial ATP-dependent K+ channel (mitoKATP) and its inward rectifier subunit (mitoKIR),” J. Biol. Chem. 279:32562-32568 (2004). The nature and sequences of these proteins, however, remain unknown.

For the foregoing reasons, there is a need to ascertain the components of mitoK_{ATP} to develop new tools for studying and influencing IPC.

### BRIEF SUMMARY

In a first aspect, an isolated SUR2A or SUR2B short form polynucleotide is summarized as including a nucleic acid sequence of SEQ ID NO:1, SEQ ID NO:3, SEQ ID NO:20 or SEQ ID NO:22. In some embodiments of the first aspect, the nucleic acid sequence is at least 90% identical to SEQ ID NO:1, SEQ ID NO:3, SEQ ID NO:20 or SEQ ID NO:22. In other embodiments of the first aspect, the nucleic acid sequence is at least 95% identical to SEQ ID NO:1, SEQ ID NO:3, SEQ ID NO:20 or SEQ ID NO:22.

In a second aspect, an isolated SUR2A or SUR2B short form polypeptide is summarized as including an amino acid sequence of SEQ ID NO:2, SEQ ID NO:4, SEQ ID NO:21 or SEQ ID NO:23. In some embodiments of the second aspect, the amino acid sequence is at least 90% identical to SEQ ID NO:2, SEQ ID NO:4, SEQ ID NO:21 or SEQ ID NO:23. In other embodiments of the second aspect, the amino acid sequence is at least 95% identical to SEQ ID NO:2, SEQ ID NO:4, SEQ ID NO:21 or SEQ ID NO:23.

In a third aspect, an expression vector that encodes a SUR2A or SUR2B short form is summarized as having a non-native expression control sequence operably linked to a non-native polynucleotide that includes a nucleic acid sequence of SEQ ID NO:1, SEQ ID NO:3, SEQ ID NO:20 or SEQ ID NO:22. In some embodiments of the third aspect, the expression vector has a non-native expression control sequence operably linked to a polynucleotide that encodes a polypeptide having an amino acid sequence of SEQ ID NO:2, SEQ ID NO:4, SEQ ID NO:21 or SEQ ID NO:23.

In a fourth aspect, a host cell comprising a non-native SUR2A or SUR2B short form not natively produced by the cell is summarized as having a non-native expression control sequence operably linked to a polynucleotide that includes a nucleic acid sequence of SEQ ID NO:1, SEQ ID NO:3, SEQ ID NO:20 or SEQ ID NO:22. In some embodiments of the fourth aspect, the host cell has a non-native expression control sequence operably linked to a non-native polynucleotide that encodes a polypeptide having an amino acid sequence of SEQ ID NO:2, SEQ ID NO:4, SEQ ID NO:21 or SEQ ID NO:23. In other embodiments of the fourth aspect, the host cell further comprises a polynucleotide that encodes a K_{IR}6.x subunit, and alternatively comprises a K_{IR}6.x polypeptide in operable interaction with the SUR2A or SUR2B short form.

In a fifth aspect, a method of screening for agents that can protect a tissue from ischemia is summarized as including the step of administering a test agent to host cells expressing a non-native SUR2A or SUR2B short form in operable interaction with a K_{IR}6.x subunit under conditions that simulate in the host cells a reduced oxygen availability condition, such as those conditions found in tissues at risk of ischemia. Prolonged cell survival in the presence of the test agent relative to survival of cells not exposed to the test agent suggests that the agent has anti-ischemic activity. In some embodiments of the fifth aspect, the SUR2A or SUR2B short forms comprise a non-native expression control sequence operably linked to a polynucleotide that includes a nucleic acid sequence of SEQ ID NO:1, SEQ ID NO:3, SEQ ID NO:20 or SEQ ID NO:22. In still other embodiments of the fifth aspect, the SUR2A or SUR2B short forms comprise a non-native expression control sequence operably linked to a polynucleotide that encodes a polypeptide having an amino acid sequence of SEQ ID NO:2, SEQ ID NO:4, SEQ ID NO:21 or SEQ ID NO:23.

In a sixth aspect, a method for identifying agents that modulate mitoK_{ATP} activity is summarized as including the steps of administering a test agent to cells that non-natively express at least one SUR2A or SUR2B short form in operable interaction with a K_{IR}6.x subunit and of evaluating mitoK_{ATP} activity. Advantageously, the operably interactive subunits are provided in the cell membrane for convenient measurement of ion channel activity. Such measurements are difficult to perform on native mitoK_{ATP} channels that are located in the inner mitochondrial membrane. It is contemplated that targeting to a cell membrane can be accomplished by altering the components of mitoK_{ATP} (i.e., the K_{IR}6.x subunit, the SUR2A short form or the SUR2B short form). Agents that modulate the activity of mitoK_{ATP} of the cells may either increase or decrease activity when compared to control cells not administered the test agent. In other embodiments of the sixth aspect, the SUR2A or SUR2B short forms comprise a non-native expression control sequence operably linked to a polynucleotide that includes a nucleic acid sequence of SEQ ID NO:1, SEQ ID NO:3, SEQ ID NO:20 or SEQ ID NO:22. In other embodiments of the sixth aspect, the SUR2A or SUR2B short forms comprise a non-native expression control sequence operably linked to a polynucleotide that encodes a polypeptide having an amino acid sequence of SEQ ID NO:2, SEQ ID NO:4, SEQ ID NO:21 or SEQ ID NO:23.

These and other features, aspects and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIG. 1 shows the structure of full-length SUR2, which has seventeen transmembrane (TM 1-17)-spanning helices, three transmembrane domains (TMD 0-2) and two nucleotide binding domains (NBD 1-2);

FIG. 2A shows the structure of SUR2A and SUR2B, as well as sites of primer synthesis; FIG. 2B shows the relationship between SUR2A and SUR2B and their respective short forms, described below, as well as sites of alternative primer synthesis;

FIG. 3 shows representative current traces recorded from cells lines containing K_{IR}6.2/NMT 55-SUR2A (A. Before intercellular perfusion; B. After intercellular perfusion; and C. After adding 100 μM ATP); and

FIG. 4 shows representative current traces recorded from cells lines containing K_{IR}6.2/NMT 55-SUR2A (A. Before intercellular perfusion; B. After intercellular perfusion; and C. After adding 100 μM ATP).

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents and alternatives falling within the spirit and scope of the invention as defined by the appended claims.

### DESCRIPTION OF PREFERRED EMBODIMENTS

The present invention relates to the inventors' observation that mice harboring a SUR2 disruption showed smaller infarct sizes than wild-type mice without IPC. This observation suggests that some forms of SUR2 may be useful in the study of the structure of the components of mitoK_{ATP} and its use in developing new tools for studying and influencing IPC.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

In describing the embodiments herein and claiming the present invention, the following terminology will be used in accordance with the definitions set out below.

As used herein, a “coding sequence” means a sequence that “encodes” a particular protein, and is a nucleic acid sequence that is transcribed (in the case of DNA) and translated (in the case of mRNA) into a polypeptide in vitro or in vivo when placed under the control of appropriate regulatory sequences. The boundaries of the coding sequence arc determined by a start codon at a 5′ (amino) terminus and a translation stop codon at a 3′ (carboxy) terminus. A coding sequence can include, but is not limited to, viral nucleic acid sequences, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, “control sequences” or “regulatory sequences” means promoter sequences, polyadenylation signals, transcription termination sequences, upstream regulatory domains, origins of replication, internal ribosome entry sites (“IRES”), enhancers, and the like, which collectively provide for replication, transcription and translation of a coding sequence in a recipient cell. Not all of these control sequences need always be present, so long as the selected coding sequence is capable of being replicated, transcribed and translated in an appropriate host cell.

As used herein, an “expression sequence” means a control sequence operably linked to a coding sequence.

As used herein, a “promoter” means a nucleotide region comprising a nucleic acid (i.e., DNA) regulatory sequence, wherein the regulatory sequence is derived from a gene that is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.) and “constitutive promoters” (where expression of a polynucleotide sequence operably linked to the promoter is unregulated and therefore continuous).

As used herein, “operably linked” means that elements of an expression sequence are configured so as to perform their usual function. Thus, control sequences (i.e., promoters) operably linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence can still be considered “operably linked” to the coding sequence.

As used herein, “operable interaction” means that subunits of a polypeptide (e.g., channels such as K_{IR}6x and SUR2A and/or SUR2B short forms), and any other accessory proteins, that are heterologously expressed in a cell assemble into a functioning (i.e., conducts a measurable voltage) channel and integrate into a cell membrane, such as a cell plasma membrane.

As used herein, “isolated polynucleotide” or “isolated polypeptide” means a polynucleotide or polypeptide isolated from its natural environment or prepared using synthetic methods such as those known to one of ordinary skill in the art. Complete purification is not required in either case. The polynucleotides and polypeptides described herein can be isolated and purified from normally associated material in conventional ways, such that in the purified preparation the polynucleotide or polypeptide is the predominant species in the preparation. At the very least, the degree of purification is such that extraneous material in the preparation does not interfere with use of the polynucleotide or polypeptide in the manner disclosed herein. The polynucleotide or polypeptide is at least about 85% pure; alternatively, at least about 95% pure; and alternatively, at least about 99% pure.

Further, an isolated polynucleotide has a structure that is not identical to that of any naturally occurring nucleic acid molecule or to that of any fragment of a naturally occurring genomic nucleic acid spanning more than one gene. An isolated polynucleotide also includes, without limitation, (a) a nucleic acid having a sequence of a naturally occurring genomic or extrachromosomal nucleic acid molecule, but which is not flanked by the coding sequences that flank the sequence in its natural position; (b) a nucleic acid incorporated into a vector or into a prokaryote or eukaryote host cell's genome such that the resulting polynucleotide is not identical to any naturally occurring vector or genomic DNA; (c) a separate molecule such as a cDNA, a genomic fragment, a fragment produced by polymerase chain reaction (PCR) or a restriction fragment; and (d) a recombinant nucleotide sequence that is part of a hybrid gene (i.e., a gene encoding a fusion protein). Specifically excluded from this definition are nucleic acids present in mixtures of clones, e.g., as these occur in a DNA library such as a cDNA or genomic DNA library. An isolated polynucleotide can be modified or unmodified DNA or RNA, whether fully or partially single-stranded or double-stranded or even triple-stranded. In addition, an isolated polynucleotide can be chemically or enzymatically modified and can include so-called non-standard bases such as inosine.

As used herein, “identical” refers those polynucleotides or polypeptides sharing at least 90% or at least 95% sequence identity to SEQ ID NOS: 1-4 and 20-23 that result in functional (i.e., associates with a K_{IR}6.x to form a K_{ATP} channel, integrates into a membrane and confers sensitivity to sulfonylureas) SUR2A or SUR2B short forms. For example, a polynucleotide or polypeptide that is at least 90% or at least 95% identical to the SUR2A and SUR2B short forms discussed below is expected to be a constituent of mitoK_{ATP}. One of ordinary skill in the art understands that modifications to either the polynucleotide or the polypeptide includes substitutions, insertions (e.g., adding no more than ten nucleotides or amino acid) and deletions (e.g., deleting no more than ten nucleotides or amino acids). These modifications can be introduced into the polynucleotide or polypeptide described below without abolishing structure and ultimately, function. Polynucleotides and/or polypeptides containing such modifications can be used in the methods of the present invention. Such polypeptides can be identified by using the screening methods described below.

An isolated nucleic acid containing a polynucleotide (or its complement) that can hybridize to any of the uninterrupted nucleic acid sequences described above, under either stringent or moderately stringent hybridization conditions, is also within the scope of the present invention. Stringent hybridization conditions are defined as hybridizing at 68° C. in 5×SSC/5× Denhardt's solution/1.0% SDS, and washing in 0.2×SSC/0.1% SDS +/−100 μg/ml denatured salmon sperm DNA at room temperature (RT), and moderately stringent hybridization conditions are defined as washing in the same buffer at 42° C. Additional guidance regarding such conditions is readily available in the art, e.g., in Sambrook et al., Molecular Cloning, A Laboratory Manual (Cold Spring Harbor Press, N.Y. 1989); and Ausubel et al. (eds.), Current Protocols in Molecular Biology, Unit 2.10 (John Wiley & Sons, N.Y. 1995).

It is well known in the art that amino acids within the same conservative group can typically substitute for one another without substantially affecting the function of a protein. For the purpose of the present invention, such conservative groups are set forth in Table 1 and are based on shared properties.

TABLE 1
Amino Acid Conservative Substitutions.

| Original Residue | Conservative Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

The invention will be more fully understood upon consideration of the following non-limiting Examples.

### EXAMPLES

### Example 1

### Isolation and Identification of SUR2A and SUR2B Short Forms from Heart Mitochondria

### Methods

Nucleotide Sequences: Sequence information for full-length SUR2A and SUR2B is located at GenBank accession numbers NM_{—}021041 and NM_{—}011511, respectively. The NCB1 database was used for exon numbering of full-length SUR2A and SUR2B cDNA.

SUR2 Mutant Mice: SUR2 mutant mice were previously described by Chutkow et al. Chutkow W, et al., “Disruption of Sur2-containing K(ATP) channels enhances insulin-stimulated glucose uptake in skeletal muscle,” Proc. Natl. Acad. Sci. USA 98:11760-11764 (2001), incorporated herein by reference as if set forth in its entirety. Briefly, a disruption cassette was inserted between exons 12 and 16 of SUR2. C57BL-6J mice (Jackson Laboratories; Bar Harbor, Me.) heterozygous for the SUR2 locus were bred into a FVB background to obtain homozygotes and genotyped. Mouse protocols and handling were performed following the guidelines of National Institutes of Health at the University Wisconsin Animal Core Facility.

K_{IR}6.1 and K_{IR}6.2 Stable Cell Lines: Cell culture and transfection were performed as described by Makielski et al. Makielski J, et al., “A ubiquitous splice variant and a common polymorphism affect heterologous expression of recombinant human SCN5A heart sodium channels,” Circ. Res. 93:821-828 (2003), incorporated herein by reference as if set forth in its entirety. Sequence information for full-length K_{IR}6.1 (CKNJ8) and K_{IR}6.2 (KCNJ11) is located at GenBank accession numbers NM_{—}008428 and NM_{—}010602, respectively. Single colonies were isolated and confirmed by RT-PCR using a SuperScript® II Kit (Iivitrogen; Carlsbad. Calif.) and Western blot analysis. Briefly, COS1 cells (1×10⁵) were seeded on a 35-mm-diameter plate in Complete Medium (Invitrogen) containing MEM (Eagle's salts and L-glutamine), 10% fetal bovine serum (FBS), 2 mM L-glutamine, 0.1 nM MEM non-essential amino acid solution, 1 mM MEM pyruvate solution, 10 U penicillin and 10 g streptomycin. 1 ug of pYB1 or pYB2 plasmid DNA containing Kir6.1 or Kir6.2 was used to transfect COS1 cells by using Superfect® Transfection Reagent (Qiagen; Valencia, Calif.), as described by Gross & Auchampach, supra. After 24 hours, the transfected cells were treated for 3 weeks with 800 μg/ml zeocin and neomycin to kill the untransfected cells.

Stable expression of K_{IR}6.1 and K_{IR}6.1 was confirmed with the primers in Table 2.

TABLE 2
K_{IR}6.1 and K_{IR}6.2 Primers.

| | |
|---|---|
| K_{IR}6.x Isoform | Primer Sequence |

| | |
|---|---|
| K_{IR}6.1 | P1: 5′-CTATCATGTGGTGGCTGGTG-3′ |
| | (SEQ ID NO:5) |
| | P2: 5′-CGTGGTTTTCTTGACCACCT-3′ |
| | (SEQ ID NO:6) |
| K_{IR}6.2 | P3: 5′-AGAATATCGTCGGGCTGATG-3′ |
| | (SEQ ID NO:7) |
| | P4: 5′-GTTTCTACCACGGCTTCCAA-3′ |
| | (SEQ ID NO:8) |

Using these primers, an expected 0.45 Kb band was observed for K_{IR}6.1, and an expected 1.1 Kb band was observed for K_{IR}6.2.

Total proteins extracted from the positive clones were subjected to Western blot analysis using anti-K_{IR}6.1 or anti-K_{IR}6.2 antibodies (Santa Cruz Biotechnology; Santa Cruz, Calif.). A band the size of ˜48 kDa was detected for K_{IR}6.1, and a band the size of ˜44 kDa was detected for K_{IR}6.2, indicating that two separate COS1 lines stably expressed K_{IR}6.1 or K_{IR}6.2.

Then, the COS1-based stable cell lines containing K_{IR}6.1 or K_{IR}6.2 were transiently transfected with SUR1, SUR2A or SUR2B to assess the specificity of the antibodies shown in Table 3. These cell lines were useful in subsequent co-expression experiments with SUR1, SUR2A or SUR2B.

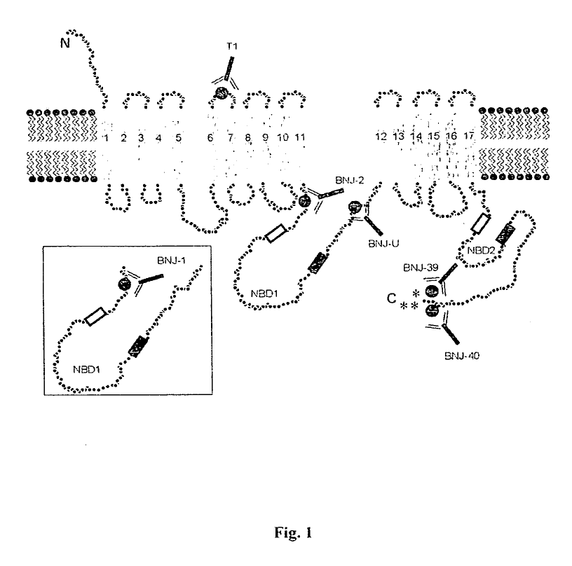

Antibodies: Table 3 shows various antibodies used to identify the novel SUR2A and SUR2B short forms from mitochondria. The binding sites of relevant antibodies are also shown in FIG. 1. A SUR1-based antibody, BNJ-1, and a SUR2-based antibody, BNJ-2, recognized SUR1 and SUR2, respectively. A third antibody, BNJ-U recognized both SUR1 and SUR2. Two antibodies, BNJ-39 and BNJ-40, recognized the C-terminus of SUR2A and SUR2B, respectively. T1 recognized SUR2. FIG. 1 shows where each antibody binds to SUR, as well as the epitope each was designed to bind.

Each epitope was synthesized by KLH-conjugation at its N-terminus and affinity-purified with a kit from Zymed (San Francisco, Calif.). As noted above, the isoform or variant specificity of each antibody was tested in K_{IR}6.2-stable cells by introducing SUR1 or SUR2.

Other antibodies used herein include the following: anti-Naᵥ1.5 (Upstate; Lake Placid, N.Y.); anti-HCN4 (Alomone Labs; Jerusalem, Israel); anti-Na/K ATPase (Abcam; Cambridge, Mass.), anti-VDAC1 (Abcam) and anti-COXIV (Abcam). Secondary antibodies were obtained from Invitrogen and Amersham (Piscataway, N.J.).

TABLE 3
SUR Antibodies.

| | N-/C- | SUR | |
|---|---|---|---|
| | Terminus | Isoform | |
| Antibody | Binding | Recognized | Epitope |
| T1 | N-terminus | SUR2 | C-YEEQKKKAADHPNRTPSIWL-N |
| | | | (SEQ ID NO:9) |
| BNJ-1 | N-terminus | SUR1 | C-VVNRKRPAREEVRD-N |
| | | | (SEQ ID NO:10) |
| BNJ-2 | N-terminus | SUR2 | C-QSKPINRKQPGRYH-N |
| | | | (SEQ ID NO:11) |
| BNJ-U | N-terminus | SUR1/SUR2 | C-HWKTLMNRQDQELE-N |
| | | | (SEQ ID NO:12) |
| BNJ-39 | C-terminus | SUR2A | C-DTGPNLLQHKNGLFSTLVMTNK-N |
| | | | (SEQ ID NO:13) |
| BNJ-40 | C-terminus | SUR2B | C-EYDTPESLLAQEDG-N |
| | | | (SEQ ID NO:14) |

Protein Extraction and Western Blot Analysis: Protein isolation was undertaken on ice or at 4° C. to prevent degradation. Crude extracts were isolated from COS1-based cells, heart or brain. Protein concentrations were determined by the Lowry method using a DC Protein Assay Kit (Bio-Rad; Hercules, Calif.). Primary antibodies were diluted 1:500-1:2000, whereas secondary antibodies were diluted 1:10,000-1:15,000. Blots were scanned with a BioSpectrum® Imaging System (UVP; Upland, Calif.).

Two-Dimensional (2D) Gel Electrophoresis: 2D gels were run as previously described by O'Farrell. O'Farrell P, “High resolution two-dimensional electrophoresis of proteins,” J. Biol. Chem. 250:4007-4021 (1975), incorporated herein by reference as if set forth in its entirety. Each protein sample was first denatured by dissolving it in SDS sample buffer. The sample was then applied to the top of a thin tube gel containing 2% ampholines, and isoelectric focusing (IEF) was carried out overnight. After a brief equilibration in SDS buffer, the tube gel was sealed to the top of a stacking gel overlaying a 10% slab gel. SDS slab gel electrophoresis was carried out for four to five hours followed by staining using Coomassie Blue R250. Polypeptides were then separated according to independent parameters of isoelectric point and molecular weight. Molecular weight standards were loaded in the 2D gel along with one IEF internal standard. A pI standard was also run in the same gel (i.e., tropomyosin, which has a doublet with pI 5.2 (MW 33 kDa)).

Isolation of a Non-Mitochondria Cell Membrane Fraction: Crude extracts were centrifuged and separated by a discontinuous sucrose gradient at 141,000×g for 2 hours, which resulted in three distinct interfaces (Fractions I-III). In both heart and brain tissue, Fraction I (with 21% glucose) was used for Western blot analysis. Plasma membrane protein was isolated from Fraction I as previously described by Balijepalli et al. Balijepalli R, et al., “Depletion of T-tubules and specific subcellular changes in sarcolemmal proteins in tachycardia-induced heart failure,” Cardiovasc Res. 59:67-77 (2003), incorporated herein by reference as if set forth in its entirety. Purity of Fraction I was determined by Western blot analysis using anti-Na/K ATPase, anti-Naᵥ1.5 and anti-HCN4 antibodies (plasma membrane markers), as well as by Western blot analysis using anti-VDAC1 (outer mitochondrial membrane marker) and anti-COXIV (inner mitochondrial membrane marker).

Isolation of Mitochondrial Fraction: Mitochondria from mouse heart or brain tissue were extracted as previously described by Sims, with modifications. Sims N, “Rapid isolation of metabolically active mitochondria from rat brain and subregions using Percoll density gradient centrifugation,” J. Neurochem. 55:698-707 (1990), incorporated herein by reference as if set forth in its entirety. Ventricular tissue from eight mouse hearts was rapidly removed and put in ice-cold Extraction Buffer A from a Mitochondria Isolation Kit (Sigmna; St. Louis, Mo.). The pieces of ventricular tissue (in 1 mm³ size, 100 mg) were treated according to the manufacturer's instructions and then homogenized using a 2-mil Teflon® homogenizer (Kontes; Vineland, N.J.; size 19). Likewise, brain tissue was rapidly removed and put into ice-cold isolation buffer (pH 7.4) containing 320 mM sucrose, 2 mM EGTA and 10 mM Trizma® base. 100-200 mg of cortical tissue was homogenized using a 2-ml Teflon® homogenizer (Kontes; size 19) in isolation buffer. The heart or brain homogenate was then brought to 5 ml with the same buffer containing 12% Percoll® solution. A discontinuous Percoll® gradient (26% and 40%) was made before loading the homogenate. The tube was then centrifuged at 30,700×g for 7 minutes, yielding a dense fraction of mitochondria. This fraction was collected and diluted 1:4 with the isolation buffer, followed by a washing step at 16,700×g for 12 minutes. The resulting pellet was then washed in a washing buffer containing 110 mM KCl, 20 mM MOPS and 1 mM EGTA (BSA was added for the brain sample) at pH 7.4 at 7,300×g for 6 minutes, and finally re-suspended in the isolation buffer (for brain samples) or storage buffer (for heart samples) from the kit.

Mitochondria were lysed with 2% CHAPS-TBS (pH 7.4) for protein concentration determination or denatured in sample buffer for protein gel electrophoresis. Purity of the mitochondrial fractions was determined by Western blot analysis, using anti-VDAC1 and anti-COXIV antibodies, as well as using anti-Na/K ATPase antibody.

Co-immunoprecipitation (Co-IP) and Two-Dimensional (2D) gel electrophoresis: Mouse heart mitochondrial proteins were used for Co-IP experiments using a Seize X-Protein-A Immunoprecipitation Kit (Pierce; Milwaukee, Wis.). BNJ-39 (10 μg) or BNJ-40 (10 μg) was used to immunoprecipitate 100 μg purified wild-type (WT) mouse heart mitochondrial proteins, which were then separated by 2D gel electrophoresis. On the other hand, BNJ-U (5 μg), BNJ-39 (5 μg), BNJ-40 (5 μg) or an equal amount of rabbit IgG was used to immunoprecipitate 50 μg purified SUR2 mutant heart mitochondrial proteins, which were then subjected to Western blot analysis. Each blot was cross-reacted with anti-K_{IR}6.1 (1:200) or anti-K_{IR}6.2 (1:200).

Nested PCR and RT-PCR: PCR reactions to amplify the splice variants and other nested fragments were carried out according to a manufacturer's protocol (Ambion; Austin, Tex.) by using 1 μl rapid amplification of cDNA ends (RACE)-ready mouse or human heart library cDNA as templates. Pfu polymerase (Stratagene; La Jolla, Calif.) was used unless indicated elsewhere. Cycling included 1 cycle of initial denaturing at 94° C. for 3 minutes; 35 cycles of 30 seconds denaturing at 94° C.; 30 second annealing at 60° C.; 5 minutes of extension at 72° C.; and a final extension cycle of 10 minutes at 72° C.

Wild-type and SUR2 mutant mouse hearts were rapidly removed and immediately frozen in liquid nitrogen. Total RNA was isolated using Trizol® reagents (Invitrogen), and mRNA was extracted by using a Trizol® mRNA Direct System (Invitrogen). RT-PCR reactions were carried out according to a manufacturer's protocol using a SuperScript® II Kit (Invitrogen).

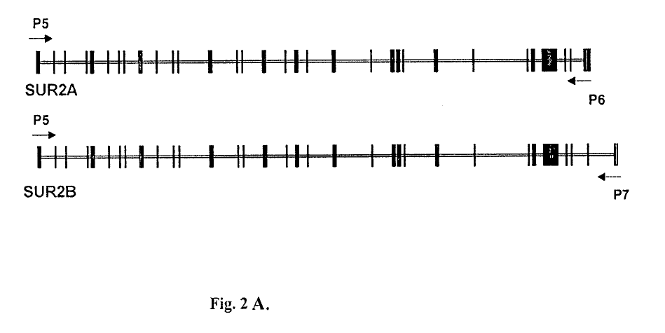

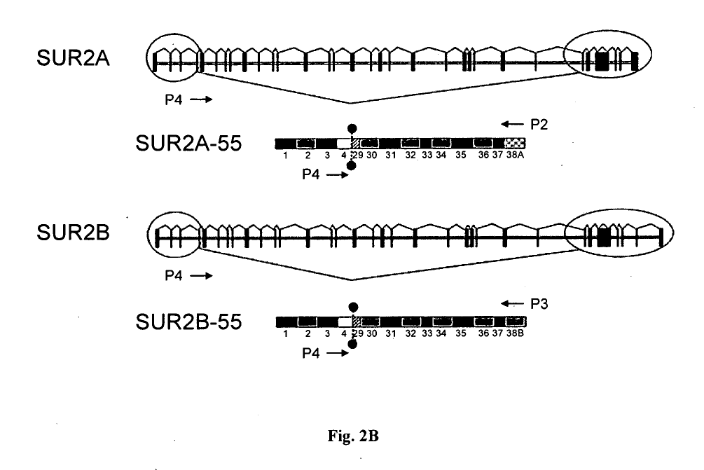

PCR was performed using a FirstChoice® RACE-Ready cDNA library generated from mouse hearts (Ambion). A 1.5-Kb PCR product was amplified from the library with primers P5 and P6 for the SUR2A short variant, or with primers P5 and P7 for the SUR2B short variant (Table 4; see also FIG. 2). To confirm the splice events in mRNA extracted from WT or SUR2 mutant mouse heart, primer P8, corresponding to nucleotide position 452-474 of the full-length SUR2, was designed (Table 4, see also FIG. 2). RT-PCR experiments were carried out with primers P6 and P8 for the SUR2A short variant (˜55 kDa) or with primers P7 and P8 for the SUR2B short variant (˜55 kDa).

TABLE 4
55 kDa SUR2A and SUR2B Primers.

| | |
|---|---|
| SUR2 IES | |
| Variant | |
| Amplified | Primer Sequence |

| | |
|---|---|
| SUR2A | P5: 5′-ATGAGCCTTTCTTTTTGTGGGAACAAC-3′ |
| (55 kDa) | (SEQ ID NO:15) |
| | P6: 5′-CTACTTGTTGGTCATCACCAAAGTA-3′ |
| | (SEQ ID NO:16) |
| | P8: 5′-AGTTGGTCAAATACTGGCAGTTG-3′ |
| | (SEQ ID NO:18) |
| SUR2B | P5: 5′-ATGAGCCTTTCTTTTTGTGGGAACAAC-3′ |
| (55 kDa) | (SEQ ID NO:15) |
| | P7: 5′-TCACATGTCTGCACGGACAAACGAGGC-3′ |
| | (SEQ ID NO:17) |
| | P8: 5′-AGTTGGTCAAATACTGGCAGTTG-3′ |
| | (SEQ ID NO:18) |

Immunocytochemistry: Immunocytochemical studies were carried out as previously described by Foell et al., with modifications. Foell J, et al., “Molecular heterogeneity of calcium channel beta-subunits in canine and human heart: evidence for differential subcellular localization,” Physiol. Genomics 17:183-200 (2004), incorporated herein by reference as if set forth in its entirety. Isolated ventricular myocytes were initially fixed with 2% paraformaldehyde in Tris-Buffered Saline (TBS, pH 7.4) for 10 minutes. Fixed cells were permeablized with 0.1% Triton® X-100 for 10 minutes and then quenched for aldehyde groups in 0.75% glycine for 10 minutes. The cells were then washed twice with TBS before incubating in 1 ml blocking solution containing 2% BSA, 2% goat serum, 0.05% NaN₃ for 2 hours at 4° C. BNJ-1 antibody (1:500) was then added to the cells while anti-rabbit Alexa Fluor-488 IgG (Invitrogen; 1:250) was added. All confocal recordings were performed at RT, and the images were analyzed by Confocal Assistant software (available on the world wide web).

Results

The antibodies identified many SUR2 proteins in mitochondrial and non-mitochondrial fractions in wild-type mice. As shown in Table 5, the SUR2 proteins ranged from 28 kDa to 150 kDa. Surprisingly, BNJ-39 and BNJ-40 identified novel ˜55 kDa SUR2A and SUR2B proteins in the mitochondrial fraction only.

TABLE 5
SUR2A and SUR2B Short Forms in Heart.

| | SUR | SUR isoforms in | |
|---|---|---|---|
| | isoform | mitochondrial | SUR isoforms in cell |
| Antibody | recognized | fraction | surface fraction |
| T1 | SUR2 | ~120 kDa | ~150 kDa |
| BNJ-2 | SUR2 | ~120 kDa | ~150 kDa |
| BNJ-U | SUR1/SUR2 | ~68 kDa | ~150 kDa |
| | | ~120 kDa | |
| BNJ-39 | SUR2A | ~28 kDa | ~28 kDa |
| | | ~55 kDa | ~68 kDa |
| | | ~68 kDa | |
| BNJ-40 | SUR2B | ~28 kDa | ~28 kDa |
| | | ~55 kDa | |

Using a combined RACE and nested PCR approach, the mRNA of the ˜55 kDa SUR2A and SUR2B short forms was isolated. When P5 and P6 or P5 and P7 were used to amplify full-length SUR2A or SUR2B from a RACE-ready cDNA library, a 1.5-Kb band was observed in addition to an expected 4.6-Kb band for full-length SUR2A and SUR2B, respectively. The 1.5-Kb SUR2A and SUR2B bands were cloned and sequenced (i.e., SEQ ID NO:1 and SEQ ID NO:3), revealing splicing variants resulting from intra-exonic splicing (IES).

IES is a rare splicing alternative characterized by splicing at non-canonical splice sites within exons in which alternative transcripts are produced. In contrast, conventional splicing is a post-transcriptional mRNA modification in which introns are removed and exons are joined. IES provides yet another level of genomic complexity which, in conjunction with intergenic splicing, significantly increases the number of predicted proteins encoded by the human genome and therefore poses challenges in deciphering genomic organization and regulation. About ten mammalian genes have been reported to contain an intra-exonic splice. This is the first ABC transporter produced by IES.

As shown in FIG. 2B, the intra-exonic splice occurs between a first point at about two-thirds of the way through exon 4 and a second point at about one-third of the way through exon 29—where CAGG from exon 29 matches the consensus motif for a 3′ IES receptor site.

To confirm that IES indeed occurred, RT-PCR was performed on mRNA isolated from wild-type mouse heart. When P8, located at nucleotide position 452-474 of full length SUR2, and P6 or P7 were used to amplify SUR2A or SUR2B, respectively, a 1.1 Kb band was observed in addition to the expected 4.2 Kb band for full-length SUR2A and SUR2B. The 1.1 Kb SUR2A and SUR2B bands were cloned and confirmed as IES variants by sequencing.

To confirm that the SUR2A and SUR2B short forms associated with K_{IR}6.x, co-immunoprecipitation was performed with the SUR2 antibodies and a heart mitochondrial fraction. Samples co-immunoprecipitated with BNJ-39 showed a ˜46 kDa spot on a 2D gel, corresponding to K_{IR}6.1. Slight variations in molecular weight of the isoforms may exist between gels because of inherent gel properties or because of different molecular weight markers, as the ˜46 kDa spot is believed to be the same as the ˜48 kDa band observed above by Western blot analysis. In addition, samples co-immunoprecipitated with BNJ-40 showed a ˜43 kDa spot on a 2D gel, corresponding to K_{IR}6.2. Likewise, the ˜43 kDa spot is believed to be the same as the ˜44 kDa band observed above by Western blot analysis. Similar results were obtained using the SUR2 mutant mice.

### Example 2

### Isolation and Identification of SUR2A and SUR2B Short Forms in Mitochondria From Brain

Methods

Protein Extraction and Western Blot analysis: Protein isolation and Western blot analysis are described above; however, instead of heart, brain was used as the tissue of interest.

Antibodies: The antibodies are described above.

Results

The antibodies identified many SUR2 proteins in mitochondrial and non-mitochondrial fractions in wild-type mice. As shown in Table 6, the SUR2 proteins ranged from 28 kDa to 160 kDa. Importantly, BNJ-39 identified the novel ˜55 kDa SUR2A protein in the plasma membrane fraction only. BNJ-40 identified the novel ˜55 kDa SUR2B protein in the mitochondrial fraction only. The short forms have an intact NBD2 and a “hybrid” TMD. The forms and locations of SUR in the brain are different from those of the heart.

TABLE 6
SUR2A and SUR2B Short Forms in Brain.

| | SUR2 | SUR2 variants in | |
|---|---|---|---|
| | variant | mitochondrial | SUR2 variants in cell |
| Antibody | recognized | membrane fraction | membrane fraction |
| BNJ-39 | SUR2A | ~68 kDa | ~160 kDa |
| | | ~28 kDa | ~55 kDa |
| BNJ-40 | SUR2B | ~55 kDa | ~150 kDa |
| | | | ~97 kDa |
| | | | ~28 kDa |

### Example 3

### Isolation of SUR2A and SUR2B Short Forms from SUR2 Mutant Mice

Methods

SUR2 Mice: SUR2 mutant mice are described above.

Protein Extraction and Western Blotting: Protein isolation and Western blot analysis are described above.

Antibodies: The antibodies are described above.

Results

The antibodies identified many SUR2 isoforms in heart from SUR2 mutant mice. As shown in Table 7, ˜55 kDa SU2A and SUR2B short forms were present in mitochondrial fractions of SUR2 mutant mice; however, no long form SUR2 was identified.

TABLE 7
SUR2 Isoforms in SUR2 Mutant Mice.

| | SUR | SUR isoforms in | |
|---|---|---|---|
| | isoform | mitochondrial | SUR isoforms in cell |
| Antibody | recognized | fraction | surface fraction |
| BNJ-39 | SUR2A | ~28 kDa | ~28 kDa |
| | | ~55 kDa (weak) | ~68 kDa |
| | | ~68 kDa | |
| BNJ-40 | SUR2B | ~55 kDa | — |

### Example 4

### Protection from Ischemia in SUR2 Mutant Mice

Methods

IPC Protocol: IPC was carried out as previously described by Kukielka et al. and Guo et al, with modifications. Kukielka G, et al., “Role of early reperfusion in the induction of adhesion molecules and cytokines in previously ischemic myocardium,” Mol. Cell. Biochem. 147:5-12 (1995); and Guo Y, et al., “Demonstration of an early and a late phase of ischemic preconditioning in mice,” Am. J. Physiol. 75:H1375-H1387 (1988), each of which is incorporated herein by reference as if set forth in its entirety. Briefly, infarctions were created in both wild-type and SUR2 mutant mice by a thirty-minute coronary artery ligature with and without IPC protocols (both acute preconditioning (APC) and delayed preconditioning (DPC; twenty-four hours after APC)). After ninety minutes of reperfusion, the size of the infarctions was determined.

Results

As shown in Table 8, SUR2 mutant mice showed significantly smaller infarctions when compared to wild-type mice, as if the SUR2 mutation conferred some protection on the mice. Interestingly, both forms of preconditioning produced only marginal improvements in infarct size in the SUR2 mutant mice. Infarction sizes were expressed as infarct area relative to area at risk for wild type. Accordingly, the SUR2 short forms appeared to autoprotect SUR2 mutant mice from ischemia, as IPC did not significantly reduce infarct size.

TABLE 8
Infarct Sizes in Wild-Type and SUR2 Mutant Mice.

| Mouse | Sham | APC | DPC |
|---|---|---|---|
| Wild-type | 37.96 ± 1.78% | 25.28 ± 1.87% | 27.40 ± 4.45% |
| SUR2 Mutant | 24.04 ± 3.73% | 18.87 ± 1.13% | 21.14 ± 2.81% |

### Example 5

### Expression of SUR2 Short Forms in Escherichia coli

Methods

E. coli expression vectors: The SUR2A and SUR2B short forms (˜55 kDa) were cloned into a pCR® Blunt II vector (Invitrogen), as pNQ52 (SUR2A short form) or pNQ57 (SUR2B short form), under the control of an inducible promoter—a Plac promoter, which is inducible with isopropyl β-D-1-thiogalactopyranoside (IPTG). Because prokaryotic cells do not recognize eukaryotic mitochondrial targeting sequences (MTS), mitochondrial proteins therefore express well in E. coli.

Subsequently, an E. coli mutant, 6106 (see, Baba T, et al., “Construction of Escherichia coli K12-in frame, single gene knockout mutants: the Keio collection,” Mol. Syst. Biol. 2:2006.0008 (2006)), in which two major K+ transport systems (ΔkdpABC5, ΔtrkA) are deleted, were transformed with K_{IR}6.2 alone, K_{IR}6.2/SUR2A (˜55 kDa) and K_{IR}6.2/SUR2B (˜55 kDa). Because of deletions in two major potassium transport systems, 6106 relies on a trkD system, which supports cell growth in low pH conditions. When grown at pH5.5 with hyper-osmotic stress (i.e., in the presence of glucose or sucrose), the 6106 mutant extrudes H+ in exchange for K+, although its initial growth is slower than wild-type, MG1655. However, transformed 6106 could show improved growth because of additional K+ transport provided by the K_{IR}6.2, K_{IR}6.2/SUR2A (5 kDa) or K_{IR}6.2/SUR2B (˜55 kDa).

Positively transformed strains, along with the untransformed 6106 and WT MG1655 (see, Blattner F, et al., “The complete genome sequence of Escherichia coli K-12,” Science 277:1453-1474 (1997)), were cultured overnight in LB medium containing 100 μg/ml ampicillin and 25 μg/ml zeocin. The cells were harvested, washed and sub-cultured into 35 ml M9 minimal medium (pH 5.5) containing 2% glucose in a 125 ml regular shake flask in the presence of antibiotics. The medium had no buffering capacity. The cultures were grown for 13 hours with a starting OD of 0.05 at 150 rpm in a 37° C. shaker.

Protein Extraction and Western Blot Analysis: Protein isolation and Western blot analysis are described above.

Antibodies: The antibodies are described above.

Results

Bacterial cultures were induced for expression of the mitoK_{ATP} proteins by adding 5 mM IPTG in the cultures for 4 hours. Total proteins were isolated and subjected to SDS-PAGE gel electrophoresis. In the IPTG-induced cultures, both SUR2A and SUR2B short form bands were observed. Western blot analysis using BNJ-39 or BNJ-40 detected each ˜55 kDa band in the pNQ52- or pNQ57-containing cells. This experiment was the first successfull heterologous expression of these short forms.

With respect to the growth experiments, WT MG1655 grew the fastest, but did not grow for more than 7 hours (i.e., once the pH in the medium dropped to around 2.0-2.2). Unlike WT MG1655, untransformed 6106 grew relatively slower, but continued to grow even when the ply in the medium dropped. However, 6106 containing K_{IR}6.2 showed a 29.8% improvement in growth when compared to untransformed 6106. Thus, a eukaryotic K+ uptake system improved growth in an E. coli K+ uptake mutant. Likewise, 6106 containing K_{IR}6.2/SUR2A (˜55 kDa) or K_{IR}6.2/SUR2B (˜55 kDa) displayed a ˜12% higher growth than 6106 containing K_{IR}6.2 alone. This data suggests that both short forms play a role in regulating K_{IR}6.2 under acidic pH conditions.

### Example 6

### Cell Lines Containing Stably Expressed SUR2 Short Forms

Methods

SUR2A and SUR2B Short Form Stable Cell Lines: Cell culture and transfections were performed as described above. Briefly, the COS1-based stable cell lines were stably transfected (although transient transfection may also be desired) with vectors encoding SUR2A (SEQ ID NO:2) and SUR2B (SEQ ID NO:4). In addition, the cells were then transiently transfected (although stable transfection may also be desired) with K_{IR}6.2 (described above). Alternatively, the cells could be transfected (stably or transiently) with K_{IR}6.1. Thus, cells lines having K_{IR}6.1/SUR2A, K_{IR}6.1/SUR2B, K_{IR}6.2/SUR2A and K_{IR}6.2/SUR2B were established. Likewise, the cell lines could contain mutated/diseased forms of K_{IR}6.1 or K_{IR}6.2. See, e.g., Hattersley T & Ashcroft F, “Activating mutations in Kir6.2 and neonatal diabetes,” Diabetes 54:2503-2513 (2005); and Bichet D, et al., “Evolving potassium channels by means of yeast selection reveals structural elements important for selectivity,” Proc. Natl. Acad. Sci. USA 101:4441-4446 (2003).

To confirm stable expression of SUR2A or SUR2B short forms, the primers in Table 4 were used. PCR was performed as described above.

Protein Extraction and Western Blot Analysis: Protein isolation and Western blot analysis are described above.

Results

Using the primer pairs described in Table 4 (i.e., P5 and P6 for SUR2A or P5 and P7 for SUR2B), a 1.5-Kb band was observed.

Using the antibodies described in Table 3 (i.e., BNJ-39 for SUR2A or BNJ-40 for SUR2B), a ˜55-kDa band was observed in the mitochondrial fraction only.

The cells expressed both a K_{IR}6.x subunit and a SUR2A or SUR2B subunit, in operable interactive relation. The operably interactive subunits were advantageously localized in the cell membrane for convenient measurement of ion channel activity.

### Example 7

### SUR2 Short Forms Lacking a Mitochondrial Targeting Sequence and Cell Lines Containing Stably Expressed SUR2 Short Forms Lacking a Mitochondrial Signaling Sequence

Methods

SUR2A and SUR2B Short Forms Lacking a Mitochondrial Targeting Sequence: The N-terminus of SUR2 contains a mitochondrial targeting sequence (MTS) motif and removal of this signal would allow the detection of a mitoK_{ATP}-like current on a cell surface. Based on a database search (including Protein Prowler Subcellular Localisation Predictor, TargetP1.1 and MitoProtII 1.0 (all available on the world wide web)), we predicted that the first 29 amino acids of the SUR2A (SEQ ID NO:2) and SUR2B (SEQ ID NO:4) short forms (i.e., MSLSFCGNNISSYNFYYYGVLQNPCFVDAL (SEQ ID NO:19)), is the MTS. We also predicted that removal of the MTS would allow expression of the short forms on a cell surface in cells that would normally target the protein to the mitochondria.

Each variant in pNQ52 or pNQ57, described above, was excised and was subcloned into a eukaryotic expression vector, pCDNA3 (Invitrogen) as pNQ55 (for the SUR2A short form) or pNQ64 (for the SUR2B short form). pNQ55 or pNQ64 were then used as templates to generate new variants in which the MTS was removed (designated as NMT 55-SUR2A (SEQ ID NOS:20-21) and NMT 55-SUR2B (SEQ ID NOS:22-23); NMT means no mitochondrial targeting sequence).

Briefly, NMT 55-SUR2A and NMT 55-SUR2B were produced using the primers in Table 9 with pNQ55 or pNQ64, respectively, as the template. PCR products were purified and cloned into a pCR®II-topo vector (Invitrogen) as pNQ78 (for NMT 55-SUR2A) or pNQ79 (for NMT 55-SUR2B). Each variant was excised and subcloned into pcDNA3 as pNQ74 (for NMT-55A) or pNQ73 (for NMT-55B) for expression in COS1 cells.

TABLE 9
NMT 55-SUR2A and NMT 55-SUR2B Primers.

| SUR2 IES | |
|---|---|
| Variants | Primer Sequence |
| NMT | P9: 5′-ATGAACCTGGTCCCACATGTCTTCCT-3′ |
| 55-SUR2A | (SEQ ID NO:24) |
| | P10: 5′-CTACTTGTTGGTCATCACCAAA-3′ |
| | (SEQ ID NO:25) |
| NMT | P11: 5′-ATGAACCTGGTCCCACATGTCTTCCT-3′ |
| 55-SUR2B | (SEQ ID NO:26) |
| | P12: 5′-TCACATGTCTGCACGGACAAACGAGGC-3′ |
| | (SEQ ID NO:27) |

NMT 55-SUR2A and NMT 55-SUR2B Short Form Stable Cell Lines: Cell culture and transfections were performed as described above, with modifications. Four stable cell lines were generated using pNQ74, pNQ73, pNQ55 and pNQ64 with COS1 cells. Briefly, COS1 cells (1×10⁵) were seeded on a 35-mm-diameter plate with Complete Medium (Invitrogen) containing MEM (Eagle's salts and L-glutamine), 10% fetal bovine serum, 2 mM L-glutamine, 0.1 nM MEM non-essential amino acid solution, 1 mM MEM pyruvate solution, 10 U penicillin and 10 g streptomycin. 1 μg of plasmid DNA of pNQ74, pNQ73, pNQ55 or pNQ64 was used to transfect COS1 cells by using the Superfect® reagents (Qiagen) as described previously. After 24 hours, transfected cells were treated with 800 μg/ml zeocin and neomycin for 3 weeks to kill untransfected cells.

Stable expression of NMT 55-SUR2A or NMT 55-SUR2B short forms was confirmed with the following primers: P13, 5′-GGAGCCAAAGCTCAAAAGTG-3′ (SEQ ID NO:28) and P14, 5′-TCFTCAGCTGGGCAATTTCT-3′ (SEQ ID NO:29). Briefly, 1×10³ transfected COS1 cells were lysed in 100 μl ddH₂O and used as templates. PCR was performed as described above, with modifications. That is, the PCR conditions included the following: 200 μM of each dNTP, 50 mM KCl, 10 mM Tris-HCl (pH 8.3), 2.0 mM MgCl₂ and 1.0 U of Taq (Promega, Madison, Wis.). The reaction mixture was subjected to a 94° C. initial denaturation for 2 minutes, followed by 35 cycles of 94° C. for 30 seconds, 50° C. for 30 seconds, 72° C. for 2 minutes, and a final extension of 72° C. for 10 minutes.

Thus, COS1-based stable cell lines containing K_{IR}^{6.2} (although K_{IR}6.1 could also be used ) were stably transfected (although transient transfection is also contemplated) with vectors encoding NMT 55-SUR2A or NMT 55-SUR2B short forms. Thus, cells lines having K_{IR}6.2/NMT 55-SUR2A and K_{IR}6.2/NMT 55-SUR2B were created. As noted above, the cell lines could contain mutated/diseased forms of K_{IR}6.1 or K_{IR}6.2

Cellular Electrophysiology: Electrophysiology experiments were carried out as previously described by Chutkow el al., with modifications. Chutkow W, et al., “Alternative splicing of sur2 Exon 17 regulates nucleotide sensitivity of the ATP-sensitive potassium channel,” J. Biol. Chem. 274:13656-13665 (1999). Briefly, K_{IR}^{6.2} was sub-cloned to 5′ of a bicistronic vector (pIRGFP, as pNQ56.) to express the desired NMT-SUR2 short forms and GFP under control of a cytomegalovirus promoter. The K_{IR}6.2 construct was transiently expressed in COS1 cell lines that stably expressed NMT 55-SUR2A or NMT 55-SUR2B. Cells expressing GFP were used for cellular electrophysiological analysis.

Potassium currents were measured at room temperature (˜22-25° C.) by an inside-out patch-clamp technique. The bath solution (cytoplasmic side) comprised the following: 140 mM KCl, 2 mM cthylene glycol-bis(β-aminioethyl ether)-N,N,N′,N′-tetraacetic acid (EGTA), 5.5 mM glucose and 5 mM N-2-hydroxyethylpiperazine-N′-2-ethanesulfonic acid (HEPES) with pH 7.3. The pH was adjusted to the designated values using KOH or HCl. 100 μM K₂ATP (Sigma) was used to test the ATP sensitivity, and the ATP-containing solutions were freshly prepared. A pipette solution (extracellular side) comprised the following: 4 mM KCl, 130 mM NaCl, 1 mM CaCl₂, 0.2 mM MgCl₂, 5 mM HEPES and 5.5 mM glucose with pH 7.4 using NaOH. Signals were recorded continuously at 0 mV using a patch-clamp amplifier (Axopatch 200; Molecular Devices; Toronto, Canada) and Clampex 10.0 software (Molecular Devices).

Results

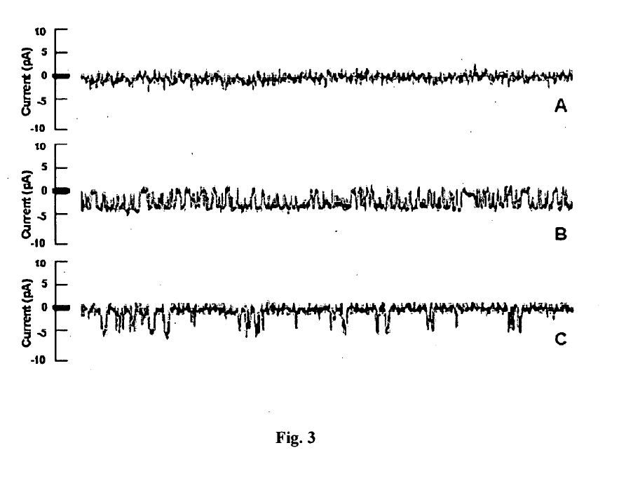

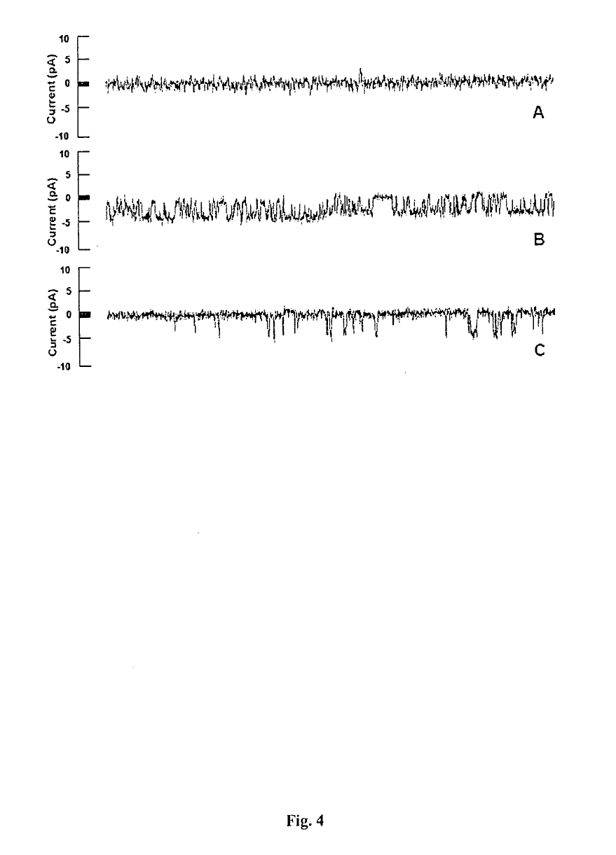

In COS1 cells containing K_{IR}6.2/NMT 55-SUR2A or K_{IR}6.2/NMT 55-SUR2B, a signature I_{K}_{_{ATP}}-like current was recorded (FIGS. 3B and 4B), which was inhibited by 100 μM ATP (FIG. 3C and 4C). Thus, the NMT 55-SUR2A and NMT 55-SUR2B short forms and channels constituted by these short forms are ATP-sensitive.

### Example 8

### (Prophetic): Identification of Cell-Protective Agents for IPC.

Methods

Cells stably expressing K_{IR}6.x (e.g., K_{IR}6.1 or K_{IR}6.2) and one of the SUR2A or SUR2B short forms (including those lacking the MTS) described herein are exposed to a test agent, e.g., an agent for which an ability to protect cells from prolonged ischemia is to be determined. Briefly, cells stably transfected with K_{IR}6.x and one of the SUR2A or SUR2B short forms described herein are exposed to the agent under in vitro conditions that simulate ischemia in vivo (e.g., hypoxia, altered ATP/ADP levels and/or metabolites of ischemia) at 37° C. By way of example only, a protocol may comprise a 30-minute equilibration phase, a 30-minute preconditioning phase (e.g., adding the agent suspected to protect cells from ischemia, such as K_{ATP} agonists), a-60 minute incubation phase, a 10-minute simulated ischemia phase (e.g., b adding 5 mM NaCN or hypoxia) and a 30-minute reperfusion phase. Following the exposure, cell viability is quantified by microscopic examination with trypan blue or by any cell viability assay known to one of ordinary skilled in the art. Control cells are incubated in similar conditions, but are not exposed to the agent.

Results

An agent that protects cells shows an increased rate of cell viability when compared to the control. In contrast, an agent that does not protect cells shows a similar or decreased rate of cell viability when compared to the control.

Alternatively, an agent that protects cells increases K_{ATP} channel activity when compared to the control. In contrast, an agent that does not protect cells decreases K_{ATP} channel activity or has similar K_{ATP} activity when compared to the control. An exemplary method of measuring K_{ATP} channel activity is patch clamping.

Known agents for altering K_{ATP} that can be used for comparison include, but are not limited to, 5-hydroxydecanoate (5-HD; channel blocker, which decreases cell viability or channel activity) or diaxozide (DIA; channel opener, which increases cell viability or channel activity) or pinacidil (PIN; channel opener, which increases cell viability or channel activity).

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

What is claimed is:

1. An isolated polynucleotide that encodes a polypeptide selected from the group consisting of SEQ ID NO:2, SEQ ID NO:4, SEQ ID NO:21 and SEQ ID NO:23 or the complement thereof.

2. The isolated polynucleotide of claim 1, wherein the polynucleotide is selected from the group consisting of SEQ ID NO:1, SEQ ID NO:3, SEQ ID NO:20 and SEQ ID NO:22.

3. An isolated polypeptide selected from the group consisting of SEQ ID NO:2, SEQ ID NO:4, SEQ ID NO:21 and SEQ ID NO:23.

4. An isolated polynucleotide that specifically hybridizes under high stringency conditions to a polynucleotide having SEQ ID NO:1, SEQ ID NO:3, SEQ ID NO:20 or SEQ ID NO:22 or a polynucleotide complementary thereto, wherein said isolated polynucleotide encodes a SUR2A or SUR2B short form, respectively.

5. An expression vector comprising a polynucleotide that encodes a polypeptide selected from the group consisting of SEQ ID NO:2, SEQ ID NO:4, SEQ ID NO:21 and SEQ ID NO:23, the polynucleotide being operably linked to an upstream expression control sequence not natively linked to the polynucleotide.

6. The expression vector of claim 5, wherein the polynucleotide is selected from the group consisting of SEQ ID NO:1, SEQ ID NO:3, SEQ D NO:20 and SEQ ID NO:22.

7. A host cell comprising a K_{IR}6.x subunit in operable interaction with a non-native SUR2A or SUR2B short form polypeptide.

8. The host cell of claim 7, comprising a polynucleotide that encodes a polypeptide selected from the group consisting of SEQ ID NO:2, SEQ ID NO:4 SEQ ID NO:21 and SEQ ID NO:23, the polynucleotide being operably linked to an upstream expression control sequence not natively linked to the polynucleotide.

9. The host cell of claim 8, wherein the polynucleotide is selected from the group consisting of SEQ ID NO:1, SEQ ID NO:3, SEQ ID NO:20 and SEQ ID NO:22.

10. A method of identifying an agent that can protect a tissue from ischemia, the method comprising the step of:
administering a test agent suspected of having cell-protective activity to host cells that comprise a K_{IR}6.x subunit in operable interaction with a SUR2A or SUR2B short form polypeptide under conditions that simulate ischemia in vitro, wherein increased cell survival in the presence of the test agent relative to the survival of cells not exposed to the test agent correlates with an ischemic protective activity of the agent.

11. A method as recited in claim 10, wherein the host cells comprise an expression vector that comprises a polynucleotide that encodes a polypeptide selected from the group consisting of SEQ ID NO:2, SEQ ID NO:4, SEQ ID NO:21 and SEQ ID NO:23.

12. A method as recited in claim 11, wherein the polynucleotide is selected from the group consisting of SEQ ID NO:1, SEQ ID NO:3, SEQ ID NO:20 and SEQ ID NO:22, and is operably linked to an upstream expression control sequence not natively linked to the polynucleotide.

13. A method for identifying an agent that modulates mitoK_{ATP} activity, the method comprising the step of:
administering a test agent to host cells that express at least one short form of SUR2A or SUR2B polypeptide and a K_{IR}6.x subunit in operable interaction, and
evaluating mitoK_{ATP} activity in cells treated with the agent relative to control cells not administered the test agent.

14. A method as claimed in claim 13, wherein the polypeptide is selected from the group consisting of SEQ ID NO:2, SEQ ID NO:4, SEQ ID NO:21 and SEQ ID NO:23.

15. A method as claimed in claim 14, wherein the cells comprise an expression vector comprising a polynucleotide that encodes the polypeptide operably linked to an upstream expression control sequence not natively linked to the polynucleotide.

16. A method as claimed in claim 15, wherein the polynucleotide is selected from the group consisting of SEQ ID NO:1, SEQ ID NO:3, SEQ ID NO:20 and SEQ ID NO:22.


SEQUENCE LISTING (29 sequences)

SEQ ID NO: 1 - DNA, 1473 bp, Mus musculus
  CDS (1)..(1473)
atgagcctttctttttgtgggaacaacatctcctcctacaacatctattatggtgttctccaaaacccctgctttgtggacgcactcaacctggtcccacatgtcttcctgctgtttatcacctttccaatactgttcattggatgggggagccaaagctcaaaagtgcaaattcatcataacacgtggcttcattttcccggacacaacctgagatggattctgacgtttgcgctcctgtttgtgcatgtttgtgagatagcggaaggcatcgtttcagactcgcatcgggcgtccaggcatctccaccttttcatgccggctgtgatgggatttgtcgccacaacaacgtccatcgtctattatcataacattgaaacatcaaatttccctaaattacttttagctttattcctgtactgggtaatggcctttattacaaagacgataaagttggtcaaatactggcagttggggtgggaggtcaggacggactatctgggagcgtgcattgttctgacagcctccattgcatccattagtggctcttccaactctggactggtgggcttgggccttctctatgccctcacgataaccaattacctaaattgggttgtaaggaacttggccgacctggaagtccagatgggtgcagtgaagaaagtgaacagtttcttgactatggagtccgagaactatgaaggcaccatggatccttctcaagtcccagagcactggccacaggaaggcgagatcaagattcacgatctgtgtgtcagatatgaaaataacctgaagcccgttctgaaacatgtcaaggcttacatcaaacccgggcagaaggtgggcatctgtggtcgaactggcagtgggaagtcctccttatccctggctttcttcagaatggtcgacatatttgacggaaagatagtcattgatgggatagacatctccaaactgcccttgcacacgctccgctccaggctgtccatcattctccaggacccaatcctgttcagcggctccatcaggtttaacctggatcctgaatgcaagtgcacagatgacaggctctgggaggctctagaaattgcccagctgaagaatatggtcaaatctctgcccggaggcttagatgccactgtcaccgaaggtggtgagaacttcagtgttggacagagacagctgttctgcctggccagggcctttgttcggaagagcagtatactcatcatggatgaagccactgcttccatcgacatggccacggaaaacattttgcagaaagtagtcatgacagcctttgcggatcgcacggtcgtaaccatagctcaccgtgtctcttctattgtggatgcaggccttgttttagtcttttctgagggtattttagtggagtgtgatactggtccaaacctgctccagcacaagaatggcctcttttctactttggtgatgaccaacaagtag

SEQ ID NO: 2 - protein, 490 aa, Mus musculus
MSLSFCGNNISSYNIYYGVLQNPCFVDALNLVPHVFLLFITFPILFIGWGSQSSKVQIHHNTWLHFPGHNLRWILTFALLFVHVCEIAEGIVSDSHRASRHLHLFMPAVMGFVATTTSIVYYHNIETSNFPKLLLALFLYWVMAFITKTIKLVKYWQLGWEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVSSIVDAGLVLVFSEGILVECDTGPNLLQHKNGLFSTLVMTNK

SEQ ID NO: 3 - DNA, 1473 bp, Mus musculus
  CDS (1)..(1473)
atgagcctttctttttgtgggaacaacatctcctcctacaacatctattatggtgttctccaaaacccctgctttgtggacgcactcaacctggtcccacatgtcttcctgctgtttatcacctttccaatactgttcattggatgggggagccaaagctcaaaagtgcaaattcatcataacacgtggcttcattttcccggacacaacctgagatggattctgacgtttgcgctcctgtttgtgcatgtttgtgagatagcggaaggcatcgtttcagactcgcatcgggcgtccaggcatctccaccttttcatgccggctgtgatgggatttgtcgccacaacaacgtccatcgtctattatcataacattgaaacatcaaatttccctaaattacttttagctttattcctgtactgggtaatggcctttattacaaagacgataaagttggtcaaatactggcagttggggtgggaggtcaggacggactatctgggagcgtgcattgttctgacagcctccattgcatccattagtggctcttccaactctggactggtgggcttgggccttctctatgccctcacgataaccaattacctaaattgggttgtaaggaacttggccgacctggaagtccagatgggtgcagtgaagaaagtgaacagtttcttgactatggagtccgagaactatgaaggcaccatggatccttctcaagtcccagagcactggccacaggaaggcgagatcaagattcacgatctgtgtgtcagatatgaaaataacctgaagcccgttctgaaacatgtcaaggcttacatcaaacccgggcagaaggtgggcatctgtggtcgaactggcagtgggaagtcctccttatccctggctttcttcagaatggtcgacatatttgacggaaagatagtcattgatgggatagacatctccaaactgcccttgcacacgctccgctccaggctgtccatcattctccaggacccaatcctgttcagcggctccatcaggtttaacctggatcctgaatgcaagtgcacagatgacaggctctgggaggctctagaaattgcccagctgaagaatatggtcaaatctctgcccggaggcttagatgccactgtcaccgaaggtggtgagaacttcagtgttggacagagacagctgttctgcctggccagggcctttgttcggaagagcagtatactcatcatggatgaagccactgcttccatcgacatggccacggaaaacattttgcagaaagtagtcatgacagcctttgcggatcgcacggtcgtaaccatagctcatcgggttcacaccattctgactgcagacctggttattgtgatgaagagaggaaatattttggaatacgacaccccggaaagcctcttggcccaggaagatggagtgtttgcctcgtttgtccgtgcagacatgtga

SEQ ID NO: 4 - protein, 490 aa, Mus musculus
MSLSFCGNNISSYNIYYGVLQNPCFVDALNLVPHVFLLFITFPILFIGWGSQSSKVQIHHNTWLHFPGHNLRWILTFALLFVHVCEIAEGIVSDSHRASRHLHLFMPAVMGFVATTTSIVYYHNIETSNFPKLLLALFLYWVMAFITKTIKLVKYWQLGWEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVHTILTADLVIVMKRGNILEYDTPESLLAQEDGVFASFVRADM

SEQ ID NO: 5 - DNA, 20 bp, Artificial
  Synthetic polynucleotide
ctatcatgtggtggctggtg

SEQ ID NO: 6 - DNA, 20 bp, Artificial
  Synthetic polynucleotide
cgtggttttcttgaccacct

SEQ ID NO: 7 - DNA, 20 bp, Artificial
  Synthetic polynucleotide
agaatatcgtcgggctgatg

SEQ ID NO: 8 - DNA, 20 bp, Artificial
  Synthetic polynucleotide
gtttctaccacgccttccaa

SEQ ID NO: 9 - protein, 20 aa, Artificial
  Synthetic polypeptide
YEEQKKKAADHPNRTPSIWL

SEQ ID NO: 10 - protein, 14 aa, Artificial
  Synthetic polypeptide
VVNRKRPAREEVRD

SEQ ID NO: 11 - protein, 14 aa, Artificial
  Synthetic polypeptide
QSKPINRKQPGRYH

SEQ ID NO: 12 - protein, 14 aa, Artificial
  Synthetic polypeptide
HWKTLMNRQDQELE

SEQ ID NO: 13 - protein, 22 aa, Artificial
  Synthetic polypeptide
DTGPNLLQHKNGLFSTLVMTNK

SEQ ID NO: 14 - protein, 14 aa, Artificial
  Synthetic polypeptide
EYDTPESLLAQEDG

SEQ ID NO: 15 - DNA, 27 bp, Artificial
  Synthetic polynucleotide
atgagcctttctttttgtgggaacaac

SEQ ID NO: 16 - DNA, 25 bp, Artificial
  Synthetic polynucleotide
ctacttgttggtcatcaccaaagta

SEQ ID NO: 17 - DNA, 27 bp, Artificial
  Synthetic polynucleotide
atgagcctttctttttgtgggaacaac

SEQ ID NO: 18 - DNA, 23 bp, Artificial
  Synthetic polynucleotide
agttggtcaaatactggcagttg

SEQ ID NO: 19 - protein, 30 aa, Artificial
  Synthetic polypeptide
MSLSFCGNNISSYNIYYYGVLQNPCFVDAL

SEQ ID NO: 20 - DNA, 1386 bp, Mus musculus
  CDS (1)..(1386)
aacctggtcccacatgtcttcctgctgtttatcacctttccaatactgttcattggatgggggagccaaagctcaaaagtgcaaattcatcataacacgtggcttcattttcccggacacaacctgagatggattctgacgtttgcgctcctgtttgtgcatgtttgtgagatagcggaaggcatcgtttcagactcgcatcgggcgtccaggcatctccaccttttcatgccggctgtgatgggatttgtcgccacaacaacgtccatcgtctattatcataacattgaaacatcaaatttccctaaattacttttagctttattcctgtactgggtaatggcctttattacaaagacgataaagttggtcaaatactggcagttggggtgggaggtcaggacggactatctgggagcgtgcattgttctgacagcctccattgcatccattagtggctcttccaactctggactggtgggcttgggccttctctatgccctcacgataaccaattacctaaattgggttgtaaggaacttggccgacctggaagtccagatgggtgcagtgaagaaagtgaacagtttcttgactatggagtccgagaactatgaaggcaccatggatccttctcaagtcccagagcactggccacaggaaggcgagatcaagattcacgatctgtgtgtcagatatgaaaataacctgaagcccgttctgaaacatgtcaaggcttacatcaaacccgggcagaaggtgggcatctgtggtcgaactggcagtgggaagtcctccttatccctggctttcttcagaatggtcgacatatttgacggaaagatagtcattgatgggatagacatctccaaactgcccttgcacacgctccgctccaggctgtccatcattctccaggacccaatcctgttcagcggctccatcaggtttaacctggatcctgaatgcaagtgcacagatgacaggctctgggaggctctagaaattgcccagctgaagaatatggtcaaatctctgcccggaggcttagatgccactgtcaccgaaggtggtgagaacttcagtgttggacagagacagctgttctgcctggccagggcctttgttcggaagagcagtatactcatcatggatgaagccactgcttccatcgacatggccacggaaaacattttgcagaaagtagtcatgacagcctttgcggatcgcacggtcgtaaccatagctcaccgtgtctcttctattgtggatgcaggccttgttttagtcttttctgagggtattttagtggagtgtgatactggtccaaacctgctccagcacaagaatggcctcttttctactttggtgatgaccaacaagtag

SEQ ID NO: 21 - protein, 461 aa, Mus musculus
NLVPHVFLLFITFPILFIGWGSQSSKVQIHHNTWLHFPGHNLRWILTFALLFVHVCEIAEGIVSDSHRASRHLHLFMPAVMGFVATTTSIVYYHNIETSNFPKLLLALFLYWVMAFITKTIKLVKYWQLGWEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVSSIVDAGLVLVFSEGILVECDTGPNLLQHKNGLFSTLVMTNK

SEQ ID NO: 22 - DNA, 1386 bp, Mus musculus
  CDS (1)..(1386)
aacctggtcccacatgtcttcctgctgtttatcacctttccaatactgttcattggatgggggagccaaagctcaaaagtgcaaattcatcataacacgtggcttcattttcccggacacaacctgagatggattctgacgtttgcgctcctgtttgtgcatgtttgtgagatagcggaaggcatcgtttcagactcgcatcgggcgtccaggcatctccaccttttcatgccggctgtgatgggatttgtcgccacaacaacgtccatcgtctattatcataacattgaaacatcaaatttccctaaattacttttagctttattcctgtactgggtaatggcctttattacaaagacgataaagttggtcaaatactggcagttggggtgggaggtcaggacggactatctgggagcgtgcattgttctgacagcctccattgcatccattagtggctcttccaactctggactggtgggcttgggccttctctatgccctcacgataaccaattacctaaattgggttgtaaggaacttggccgacctggaagtccagatgggtgcagtgaagaaagtgaacagtttcttgactatggagtccgagaactatgaaggcaccatggatccttctcaagtcccagagcactggccacaggaaggcgagatcaagattcacgatctgtgtgtcagatatgaaaataacctgaagcccgttctgaaacatgtcaaggcttacatcaaacccgggcagaaggtgggcatctgtggtcgaactggcagtgggaagtcctccttatccctggctttcttcagaatggtcgacatatttgacggaaagatagtcattgatgggatagacatctccaaactgcccttgcacacgctccgctccaggctgtccatcattctccaggacccaatcctgttcagcggctccatcaggtttaacctggatcctgaatgcaagtgcacagatgacaggctctgggaggctctagaaattgcccagctgaagaatatggtcaaatctctgcccggaggcttagatgccactgtcaccgaaggtggtgagaacttcagtgttggacagagacagctgttctgcctggccagggcctttgttcggaagagcagtatactcatcatggatgaagccactgcttccatcgacatggccacggaaaacattttgcagaaagtagtcatgacagcctttgcggatcgcacggtcgtaaccatagctcatcgggttcacaccattctgactgcagacctggttattgtgatgaagagaggaaatattttggaatacgacaccccggaaagcctcttggcccaggaagatggagtgtttgcctcgtttgtccgtgcagacatgtga

SEQ ID NO: 23 - protein, 461 aa, Mus musculus
NLVPHVFLLFITFPILFIGWGSQSSKVQIHHNTWLHFPGHNLRWILTFALLFVHVCEIAEGIVSDSHRASRHLHLFMPAVMGFVATTTSIVYYHNIETSNFPKLLLALFLYWVMAFITKTIKLVKYWQLGWEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVHTILTADLVIVMKRGNILEYDTPESLLAQEDGVFASFVRADM

SEQ ID NO: 24 - DNA, 25 bp, Artificial
  Synthetic polynucleotide
atgaacctggtcccacatgtcttcc

SEQ ID NO: 25 - DNA, 22 bp, Artificial
  Synthetic polynucleotide
ctacttgttggtcatcaccaaa

SEQ ID NO: 26 - DNA, 26 bp, Artificial
  Synthetic polynucleotide
atgaacctggtcccacatgtcttcct

SEQ ID NO: 27 - DNA, 27 bp, Artificial
  Synthetic polynucleotide
tcacatgtctgcacggacaaacgaggc

SEQ ID NO: 28 - DNA, 20 bp, Artificial
  Synthetic polynucleotide
ggagccaaagctcaaaagtg

SEQ ID NO: 29 - DNA, 20 bp, Artificial
  Synthetic polynucleotide
tcttcagctgggcaatttct